(19) United States
(12) Patent Application Publication
(10) Pub. No.: US 2024/0268629 A1
(43) Pub. Date: Aug. 15, 2024

(54) FOOT CONTROLLER AND SURGERY ASSISTANCE DEVICE

(71) Applicant: RIVERFIELD INC., Tokyo (JP)

(72) Inventors: Tatsuhito FUKUDA, Tokyo (JP); Koh-hei SONODA, Fukuoka City (JP)

(73) Assignee: RIVERFIELD INC., Tokyo (JP)

(21) Appl. No.: 18/646,988

(22) Filed: Apr. 26, 2024

Related U.S. Application Data
(63) Continuation of application No. PCT/JP2022/021762, filed on May 27, 2022.

(30) Foreign Application Priority Data
Oct. 27, 2021 (JP) 2021-175185

(51) Int. Cl. A61B 1/00; A61B 17/00

(52) U.S. Cl. CPC A61B 1/00048; A61B 2017/00199; A61B 2017/00973

(57) ABSTRACT

A foot controller for operating of a device includes: a placement section on which a foot is to be placed; a first operating part configured to be moved back and forth and left and right by the foot placed at a first position of the placement section; and a second operating part configured to be moved back and forth and left and right by the foot placed at a second position of the placement section. The first operating part and the second operating part are arranged along a front-back direction on the placement section, and configured to select a predetermined operation of the device based on a movement of the foot.

### CROSS-REFERENCE TO RELATE APPLICATIONS

This application is a continuation of International Application No. PCT/JP2022/021762, filed on May 27, 2022, claiming priority from Japanese Patent Application No. 2021-175185, filed on Oct. 27, 2021 in the Japan Patent Office, the contents of each of which being incorporated by reference herein in their entireties.

### BACKGROUND

The present disclosure relates to a foot controller for operation and control of medical surgical tools during surgery.

For various surgical operations, pedals and foot controllers for operators, such as doctors and assistants, have been used. Operators may use the pedals and foot controllers when the operators use surgical tools to perform parts of an operation and they may control surgical tools with their foot SUMMARY

It is an aspect to provide a novel technology for an improved foot controller that is both space-saving and user-friendly.

According to an aspect of one or more embodiments, there is provided a foot controller for operating of a device includes: a placement section on which a foot is to be placed; a first operating part configured to be moved back and forth and left and right by the foot placed at a first position of the placement section; and a second operating part configured to be moved back and forth and left and right by the foot placed at a second position of the placement section. The first operating part and the second operating part are arranged along a front-back direction on the placement section, and configured to select a predetermined operation of the device based on a movement of the foot.

According to another aspect of one or more embodiments, there is provided a surgery assistance device including: an endoscope including a camera; a holder configured to hold the endoscope; an arm configured to adjust a position of the holder; a display configured to display a visual field image taken by the camera; the foot controller described above; and a controller configured to change the visual field image based on an operation on the foot controller.

According to another aspect of one or more embodiments, there is provided a surgery assistance device including: an endoscope; a holder configured to hold the endoscope; an arm configured to adjust a position of the holder; a retractor configured to retract the endoscope from an eyeball; and the foot controller described above. The retractor is configured to retract the endoscope from the eyeball corresponding to the third operating part on the foot controller being operated.

According to another aspect of one or more embodiments, there is provided a foot controller for operating a device, the foot controller including: a placement section on which a foot is to be placed; a first joystick configured to be moved back and forth and left and right by the foot placed at a first position of the placement section; and a second joystick configured to be moved back and forth and left and right by the foot placed at a second position of the placement section. The first joystick and the second joystick are arranged along a front-back direction on the placement section. The first joystick and the second joystick are further configured to select a predetermined operation of the device based on a movement of the foot.

### BRIEF DESCRIPTION OF DRAWINGS

FIG. 1A is a top view schematically illustrating a configuration of a surgical system according to some embodiments.

FIG. 1B is a side view schematically illustrating the configuration of the surgical system according to some embodiments.

FIG. 2 is a diagram schematically illustrating a cross section structure of an eyeball.

FIG. 3 is a schematic diagram illustrating a surgery assistance device according to some embodiments.

FIG. 4 is a side view of an endoscope inserted in an eyeball by a retractor according to some embodiments.

FIG. 5 is a side view of the endoscope retracted from the eyeball by the retractor according to some embodiments.

FIG. 6 is a perspective view of a foot controller according to the embodiment.

FIG. 7A is a top view of the foot controller according to some embodiments.

FIG. 7B is a side view of the foot controller according to some embodiments.

FIG. 8A is a perspective view of the foot controller in a state in which a foot is placed at a first position of a placement section according to some embodiments.

FIG. 8B is a side view of the foot controller in the state in which the foot is placed at the first position of the placement section according to some embodiments.

FIG. 9A is a perspective view of the foot controller in a state in which a foot is placed at a second position of the placement section according to some embodiments.

FIG. 9B is a side view of the foot controller in a state in which the foot is placed at the second position of the placement section according to some embodiments.

FIG. 10 is a top view of the foot controller in a state in which a foot is placed on the placement section according to some embodiments.

FIG. 11 is a block diagram of a surgery assistance device according to some embodiments.

FIG. 12A is a schematic diagram illustrating an operation of a first joystick according to some embodiments.

FIG. 12B is a schematic diagram of a visual field image corresponding to the operation of the first joystick illustrated in FIG. 12A according to some embodiments.

FIG. 13A is a schematic diagram illustrating an operation of a second joystick according to some embodiments.

FIG. 13B is a schematic diagram of a visual field image corresponding to the operation of the second joystick illustrated in FIG. 13A according to some embodiments.

### DETAILED DESCRIPTION

As discussed above, for various surgical operations, pedals and foot controllers for operators, such as doctors and assistants, have been used. For example, operators may use the pedals and foot controllers to operate the surgical tools to perform parts of an operation.

When an operation is performed with a foot controller, up to two operation modes can be selected with one switch. Thus, more switches will be needed for enabling selection of more operation modes, which makes the foot controller have a more complicated structure and a larger size. The position of the foot that presses the switch to switch the mode needs to be changed, which increases complexity.

It is an aspect to provide a novel technology for improving a foot controller to be both space saving and user-friendly.

According to some embodiments, a foot controller for operating of a device may include a placement section on which a foot is to be placed; a first operating part configured to be moved back and forth and left and right by the foot placed at a first position of the placement section; and a second operating part configured to be moved back and forth and left and right by the foot placed at a second position of the placement section. The first operating part and the second operating part are arranged along a front-back direction on the placement section, and configured to select a predetermined operation of the device based on a movement of the foot.

Accordingly, the foot controller can have a reduced width. The foot controller can be more user-friendly because the number of operations of the device with two operating parts.

According to some embodiments, the first operating part may be located at a recess on the placement section, and the second operating part may be located at a position adjacent to the foot placed at the first position.

As a result, it is unlikely that the second operating part is erroneously touched while the first operating part is being operated by the foot.

According to some embodiments, the second operating part may be located on the placement section at an elevated position from the first operating part, and the first operating part may be located at a position adjacent to the foot placed at the second position.

As a result, it is unlikely that the first operating part is erroneously touched while the second operating part is being operated by the foot.

According to some embodiments, the foot controller may further include a third operating part on a side of the placement section, the third operating part may be configured to be operated by a horizontal movement of the foot placed at the first position or by the horizontal movement of the foot placed at the second position.

Thus, the third operating part can be operated by a horizontal movement of a foot both in the case where the foot is placed at the first position and in the case where the foot is placed at the second position.

According to some embodiments, the third operating part may be a pair of switches on respective sides of the placement section, and the first operating part and the second operating part may be located in a region between the pair of switches.

Thus, a switch can be operated by a horizontal movement of the foot in either direction, which enables an operation without a sense of discomfort as compared with a case where a switch is provided only on one side of the placement section even if there is a difference in easiness of horizontal movement of a foot or a difference in easiness between a rightward movement and a leftward movement of a foot depending on the operator's dominant foot. This allows the foot controller to be operated by both of left and right feet.

According to some embodiments, the first operating part and the second operating part may be joysticks. This allows relatively easy back and forth and left and right operations with a sole.

According to some embodiments, the placement section may have a length less than 400 mm in the front-back direction, the placement section may have a width less than 250 mm, the placement section may have a height greater than 100 mm and less than 150 mm at a position having a highest elevation, and the elevated position of the placement section may be tilted at an angle greater than 100 and less than 15°.

According to another aspect of one or more embodiments, provided is provided a surgery assistance device including: an endoscope including a camera; a holder configured to hold the endoscope; an arm configured to adjust a position of the holder; a display configured to display a visual field image taken by the camera; the foot controller described above; and a controller configured to change the visual field image based on an operation on the foot controller.

Because the visual field image can be changed in accordance with the operation on the foot controller, interruption of manual work of the operator to change the visual field image can be avoided.

According to some embodiments, the controller may include a drive controller configured to move the endoscope in a back and forth direction and left and right direction, through the arm, corresponding to a movement of the first operating part.

Thus, operation of the second operating part is only needed to cause the visual field to zoom in and out without regard to the movement of the endoscope, and the visual field image can be turned by image processing without moving the endoscope.

According to some embodiments, controller may include a drive controller configured to advance and retract the endoscope, through the arm, corresponding to a back and forth movement of the second operating part by the foot, and an image processor configured to perform image processing and rotate the visual field image corresponding to a left and right movement of the second operating part by the foot. The visual field image of the endoscope may be configured to be zoom in and out of in accordance to the back and forth movement of the second operating part by the foot.

According to another aspect of one or more embodiments, there is provided a surgery assistance device including: an endoscope; a holder configured to hold the endoscope; an arm configured to adjust a position of the holder; a retractor configured to retract the endoscope from an eyeball; and the foot controller described above. The retractor is configured to retract the endoscope from the eyeball corresponding to the third operating part on the foot controller being operated.

Thus, in an emergency, the operator only needs to move his/her foot horizontally to retract the endoscope from an eyeball, for example.

Various embodiments will now be described with reference to the drawings. Components, members, and processes that are the same as or equivalent to each other illustrated in the drawings are represented by the same reference numerals, and redundant explanation will not be repeated where appropriate for conciseness. The present disclosure is not to limit the various embodiments described herein, but rather the various embodiments are provided as examples, and any feature or any combination of features described in the various embodiments is not necessarily essential.

### [Surgical System]

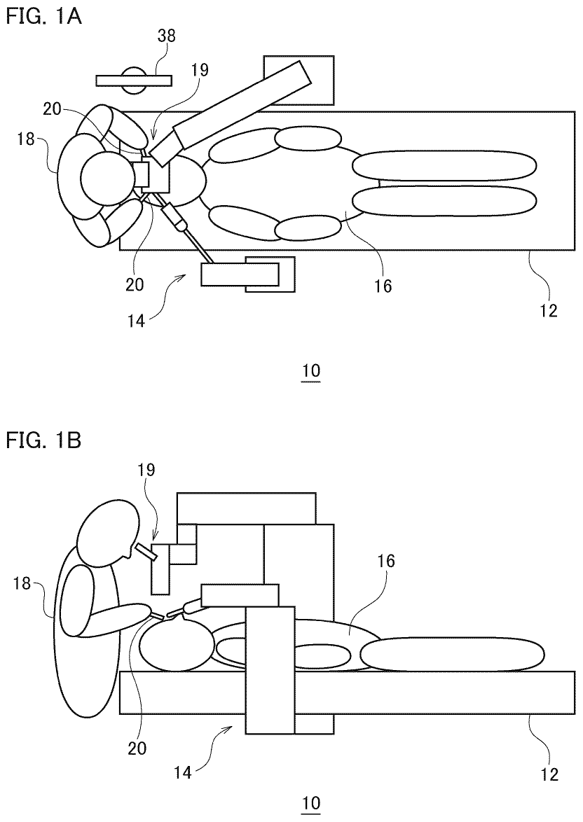

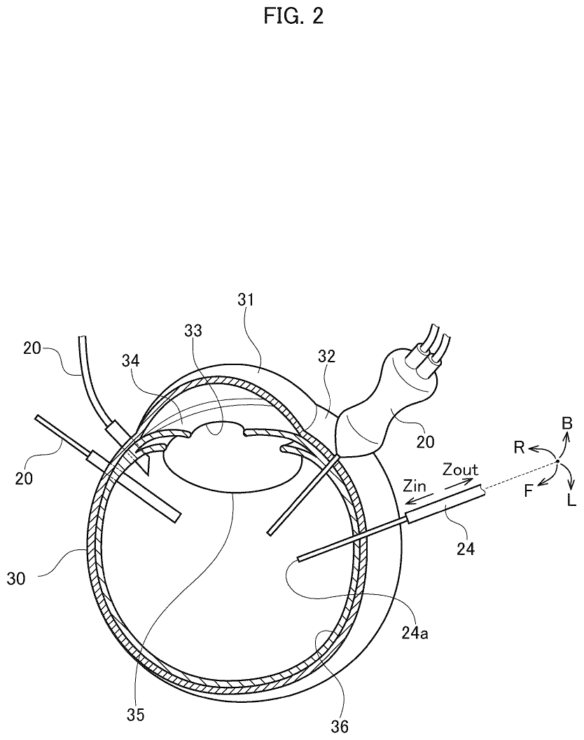

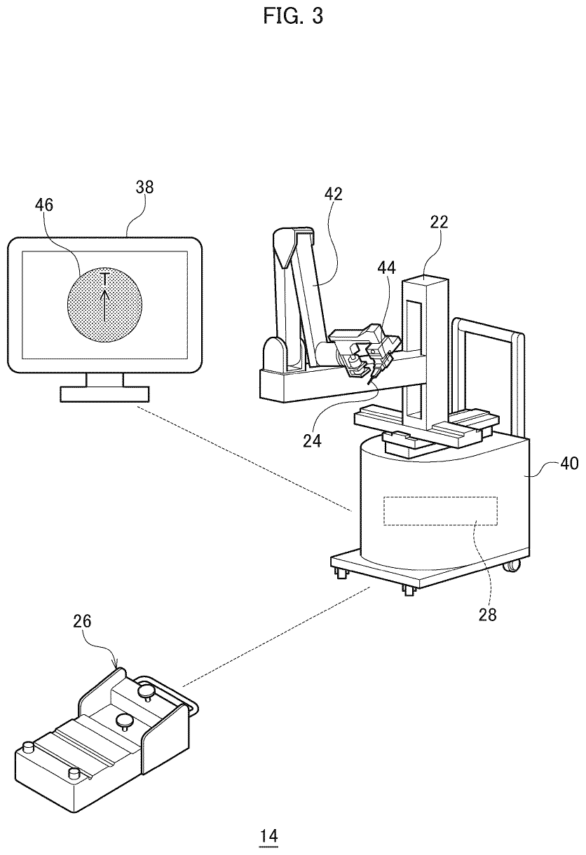

Various embodiments will be described below with reference to an example of a surgical system to be used in intraocular surgery. FIG. 1A is a top view schematically illustrating a configuration of the surgical system according to some embodiments, and FIG. 1B is a side view schematically illustrating the configuration of the surgical system according to some embodiments. FIG. 2 is a diagram schematically illustrating a cross section structure of an eyeball. FIG. 3 is a schematic diagram illustrating a surgery assistance device according to some embodiments.

A surgical system 10 illustrated in FIGS. 1A and 1B includes an operating table 12, and a surgery assistance device 14. A patient 16, who is a subject of an operation, is laid on his/her back on the operating table 12. A doctor 18, who may be a surgeon, takes his/her position at the head of the patient 16, and performs an operation inside an eyeball 30 (see FIG. 2) of the patient 16 by using an operating microscope 19 and various treatment tools 20. The treatment tools 20 may include a vitreous cutter, forceps, and an injector for irrigating solution or the like.

The surface of the eyeball 30 illustrated in FIG. 2 is covered with a cornea 31 and a conjunctiva 32, an iris 34 with a pupil 33 behind the cornea 31, and a crystalline lens 35 behind the iris 34. A retina 36 is present over an eye-ground inside the eyeball 30. The doctor 18 performs an operation inside the eyeball 30 by inserting a treatment tool 20 into the conjunctiva 32.

### [Surgery Assistance Device]

The operation on the eyeball 30 performed by the doctor 18 is assisted by the surgery assistance device 14. The surgery assistance device 14 illustrated in FIG. 3 includes an endoscope holder 22, an endoscope 24, a foot controller 26, a controller 28, and a monitor 38, which is a display. The endoscope holder 22 includes a base part 40 and an arm 42. The base part 40 is placed on a floor of an operating room or the like, and the arm 42 is attached to the base part 40. The arm 42 is rotatably supported by the base part 40.

The arm 42 may include one or more joint portions and rotatable portions and may be capable of moving a holder 44 at an arm distal end to an intended position with an intended posture. This enables control of the movement of the endoscope 24 held by the holder 44 through the arm 42. Thus, the endoscope 24 may be freely moved to any intended position by moving the arm 42 with the endoscope 24 fixed to the holder 44.

The endoscope 24 inserted in the eyeball 30 of the patient 16 may be held by the holder 44, which eliminates the need for the doctor 18 to hold the endoscope 24 with his/her hand. The doctor 18 can therefore use both of his/her hands to perform the operation on the eyeball 30.

The endoscope 24 of the surgery assistance device 14 may be inserted into the eyeball 30 in a state in which the endoscope 24 is fixed to the holder 44 (see FIG. 2). The inside of the eyeball 30 is imaged by the inserted endoscope 24. The endoscope 24 may include an image sensor, such as a camera. Image signals obtained by the image sensor of the endoscope 24 may be converted through analog-to-digital conversion into digital image signals (image data) representing luminance of a predetermined number of gradations in units of pixels. An image based on the image data from the endoscope 24 is displayed on the monitor 38.

The foot controller 26 is an operation device for operating the movement of the endoscope 24 through the arm 42. The operations of the endoscope 24 from use of the foot controller 26 may include, but are not limited to, zooming in, zooming out, and rotating an image taken by the endoscope 24 and displayed on the monitor 38, and the like. The foot controller 26 may include a joystick, a trackball, and a D-pad for operating the movement of the endoscope 24.

The controller 28 may perform various processes necessary for implementing the surgery assistance device 14 according to various embodiments, such as controlling the operation of the arm 42, generating images to be displayed on the monitor 38, and controlling the display on the monitor 38.

The controller 28 may include a microcomputer including a central processing unit (CPU), a read only memory (ROM), a random access memory (RAM), and the like. The controller 28 may be constituted by one or more microcomputers. The controller 28 may be included in the base part 40 of the endoscope holder 22. Alternatively, the controller 28 may be included in another external device.

The monitor 38 may display a visual field image 46 of the endoscope 24 based on the display control performed by the controller 28. A direction T toward the top is a direction opposite the gravitational direction, and the upward direction of the visual field image 46 on the monitor 38 may be adjusted to correspond to the direction T toward the top at initial settings.

### [Retractor]

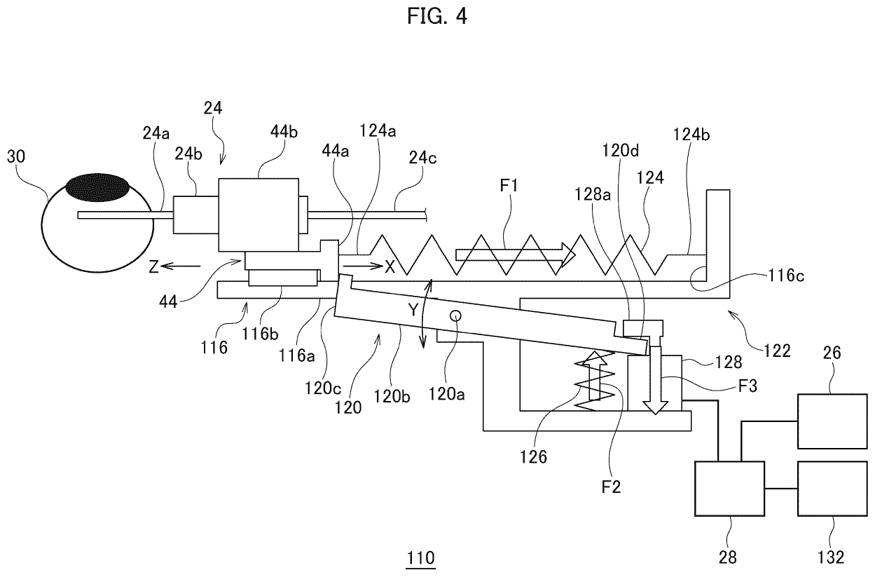

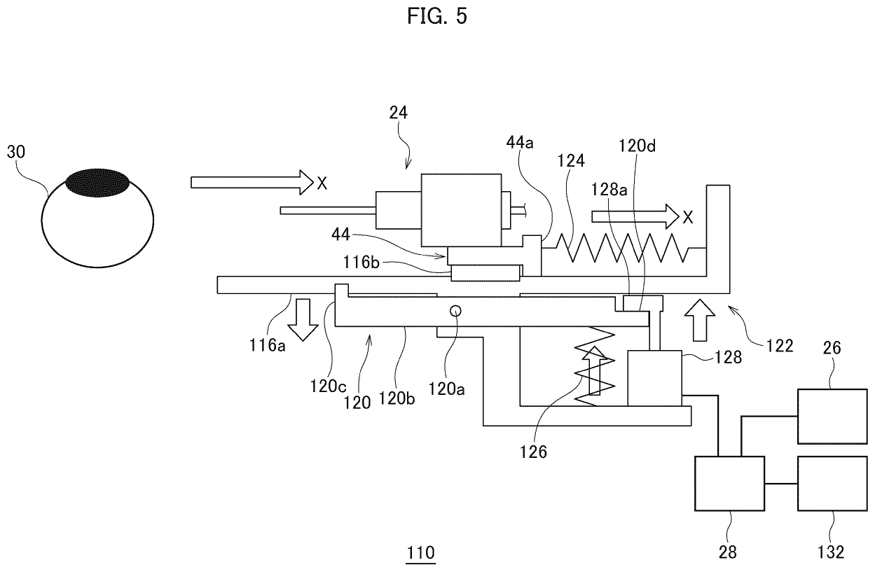

Next, a retractor for retracting the endoscope inserted in the eyeball in an emergency will be described. FIG. 4 is a side view of an endoscope inserted in an eyeball by a retractor according to some embodiments. FIG. 5 is a side view of the endoscope retracted from the eyeball by the retractor according to some embodiments. Note that the retractor according to various embodiment is provided between the endoscope 24 and the arm 42.

The retractor 110 illustrated in FIG. 4 may include the holder 44 for holding the endoscope 24, which is a surgical tool, a guide assembly 116 for guiding the holder 44 to linearly advance and retract, a lock 120 for locking the position of the holder 44 in a state in which the endoscope 24 is inserted in an eyeball 30, which is a surgical site, and a lock release 122 for releasing the lock 120 and retracting the holder 44 in accordance with a signal from outside.

The endoscope 24 may include a fiber-like insert 24a, a grip 24b, and a cable 24c. The fiber like insert may have a diameter of about 0.5 to 1.2 mm and may be inserted into an eyeball 30. The grip 24b may held by an adapter 44b of the holder 44. The guide assembly 116 may include a linear guide 116a, and a stage 116b configured to slide along the linear guide 116a.

The lock release 122 may include a tension spring 124 that may generate a first force F1 in a direction X in which the holder 44 is to be retracted in a state in which the holder 44 is locked. The lock release 122 may further include a compression spring 126 that may generate a second force F2 for releasing the locking state of the lock 120, and a pull-type solenoid 128 that may generate a third force F3 for maintaining, against the second force F2, the locking state of the lock 120.

A first end 124a of the tension spring 124 may be fixed to an end 44a in the retracting direction of the holder 44. A second end 124b of the tension spring 124 may be fixed to an end 116c of the linear guide 116a. The lock 120 may be a rod 120b configured to turn about a fulcrum 120a in a Y direction. The rod 120b has a first end 120c that comes in contact with the end 44a of the holder 44 and a second end 120d on which the third force F3 acts in the locking state.

The pull-type solenoid 128 may pull the second end 120d of the rod 120b while power is on. Specifically, the pull-type solenoid 128 has a hook 128a at an end of a plunger thereof. The hook 128a may fix the second end 120d of the rod 120b in a state in which the second end 120d is pulled downward.

The compression spring 126 of the lock release 122 according to some embodiment has a spring constant and a length set so that the locking state of the lock 120 may be released while power supply to the pull-type solenoid 128 is off. In addition, the lock release 122 may further include a controller 28 for controlling power supply to the pull-type solenoid 128, a sensor 132 for detecting the movement of the eyeball 30 or the movement of the head, and the foot controller 26.

Next, an operation of immediately retracting the endoscope 24 from the eyeball 30 in an X direction will be explained with reference to FIG. 5. As illustrated in FIG. 5, when the pull-type solenoid 128 is powered off, the third force F3 by which the hook 128a of the pull-type solenoid 128 has pulled the second end 120d of the rod 120b downward may be eliminated. As a result, the second end 120d may be pushed upward and the first end 120c, which is located on a side opposite the second end 120d with respect to the fulcrum 120a, may be pushed downward by the restoring force of the compression spring 126, and the locking of the holder 44 by the lock 120 may be released.

As a result, the restoring force of the tension spring 124 with the first end 124a fixed to the end 44a of the holder 44 may move the holder 44 in the X direction, and the endoscope 24 may be retracted from the eyeball 30 in the X direction.

As described above, the retractor 110 according to some embodiment may allow the endoscope 24 linearly inserted in the eyeball 30, which is the surgical site, along the linear guide 116a to be retracted in parallel direction (i.e. the X direction) with the inserting direction Z of the endoscope 24. As a result, the load applied to the eyeball 30 while the endoscope 24 is retracted from the eyeball 30 may be reduced.

In addition, the retractor 110 may achieve holding and retracting the endoscope 24 by adjusting the forces generated by the tension spring 124, the compression spring 126, and the pull-type solenoid 128 by a simple structure.

In addition, in the retractor 110, because the pull-type solenoid 128 requires power supply and a relatively large space can be placed at a position away from the holder 44, the flexibility of a layout of respective components is increased without making the structure complicated.

Furthermore, in such an emergency in which power supply to the pull-type solenoid 128 is cut off owing to a power failure or a device failure, the retractor 110 is capable of quickly retracting the endoscope 24 from the eyeball 30 without requiring special control. In addition, the retractor 110 is capable of generating the second force F2 by the restoring force of the compression spring 126 without using power, and similarly capable of generating the first force F1 by the restoring force of the tension spring 124 without using power.

Furthermore, in an emergency, the retractor 110 is capable of quickly retracting the endoscope 24 inserted in such a soft and delicate surgical site as the eyeball 30 while reducing the load applied to the eyeball 30.

In addition, the controller 28 of the lock release 122 according to come embodiments may be configured to release the locking state of the lock 120 by controlling a current to turn off power supply to the pull-type solenoid 128 in accordance with a signal in response to a movement of the eyeball or the like detected by the sensor 132. This allows the endoscope 24 held in a state inserted in the eyeball 30 to be quickly retracted before a large load is applied to the eyeball 30 by the movement of the position of the eyeball 30.

While the lock release 122 according to some embodiments may be a combination of the compression spring 126, which is the second force generating member, and the pull-type solenoid 128, which is the third force generating member, a lock release 122 may be configured as a combination of a tension spring, as a second force generating member, for pulling the rod 120b upward, and a push-type solenoid, as a third force generating member, for pushing the second end 120d of the rod 120b downward when power is supplied to the push-type solenoid.

### [Foot Controller]

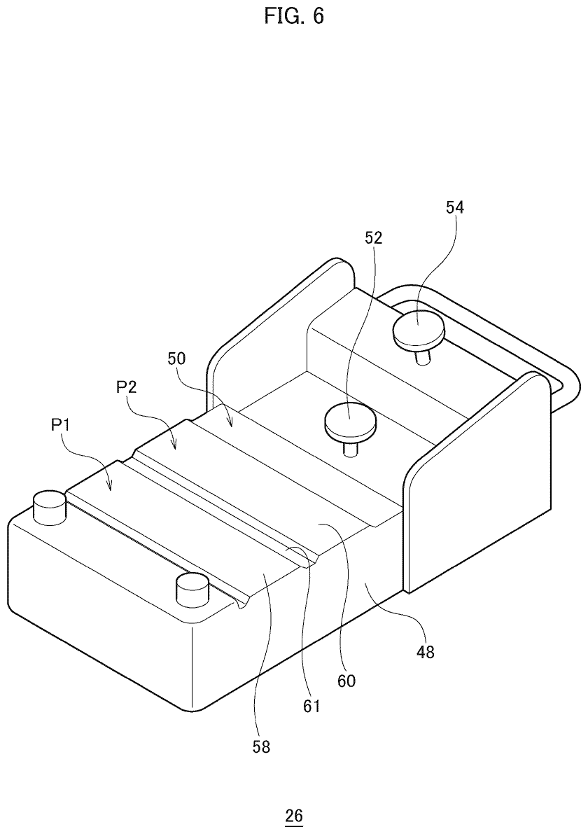

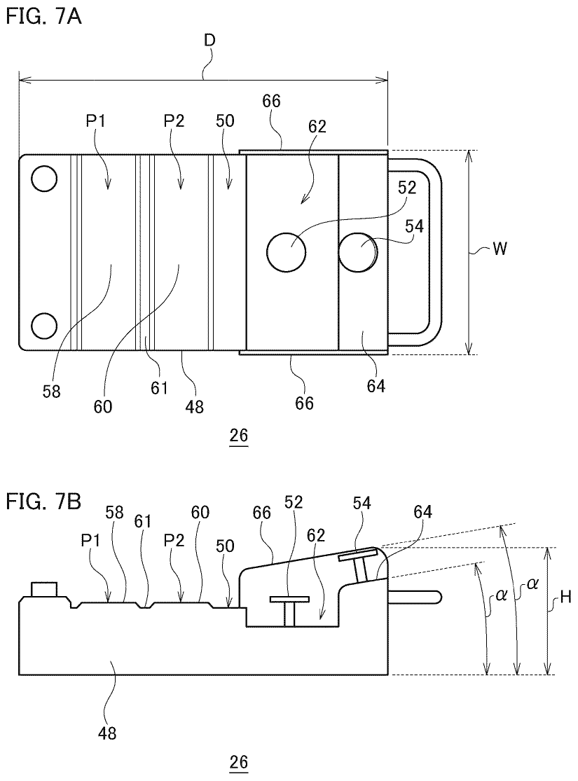

Next, a foot controller according to some embodiments will be described in detail. FIG. 6 is a perspective view of a foot controller according to some embodiments. FIG. 7A is a top view of the foot controller according to some embodiments, and FIG. 7B is a side view of the foot controller according to some embodiments. Note that illustration of a kick switch (to be described later) on a right side face of the foot controller is omitted in FIG. 7B.

The foot controller 26 includes a box-shaped housing 48, and may be used for operating the endoscope holder 22. Specifically, the foot controller 26 may include, on a top face of the housing 48, a placement section 50 on which a foot is to be placed, a first joystick 52, which is a first operating part, that may be operated back and forth and left and right by a foot placed at a first position P1 of the placement section 50, and a second joystick 54, which is a second operating part, that may be operated back and forth and left and right by a foot placed at a second position P2 of the placement section 50. The joysticks 52 and 54 may be relatively easily operated back and forth and left and right by a sole of a foot.

The first joystick 52 and the second joystick 54 may be arranged along the front-back direction on the placement section 50, and a predetermined operation of the endoscope holder 22 may be selected in accordance with the direction of the operation on the first joystick 52 or the second joystick 54. While each joystick according to some embodiments is capable of being operated in at least four directions, the joysticks may be operating parts capable of being operated in more directions.

The length D in the front-back direction of the placement section 50 is preferably larger than the foot length of a typical adult man and smaller than 400 mm. This minimizes the length D in the front-back direction of the foot controller 26. In addition, because the two joysticks are arranged along the front-back direction, the width W of the foot controller 26 can be minimized. The width W is preferably larger than the foot width of a typical adult man and smaller than 250 mm. The height H is about 100 to 150 mm at a highest position of the foot controller 26.

Because a number of operations of the endoscope holder 22 can be selected with two joysticks, the user-friendliness of the foot controller 26 according to some embodiments can be improved and the size of the entire foot controller 26 can be reduced. Note that the back and forth and left and right operations of each joystick need not necessarily be set in such a manner that each of all the operations of the joystick is associated with selection of a predetermined operation of the endoscope holder 22, but some operations may be invalid, that is, some operations the joystick may be associated with none of the operations of the endoscope holder 22.

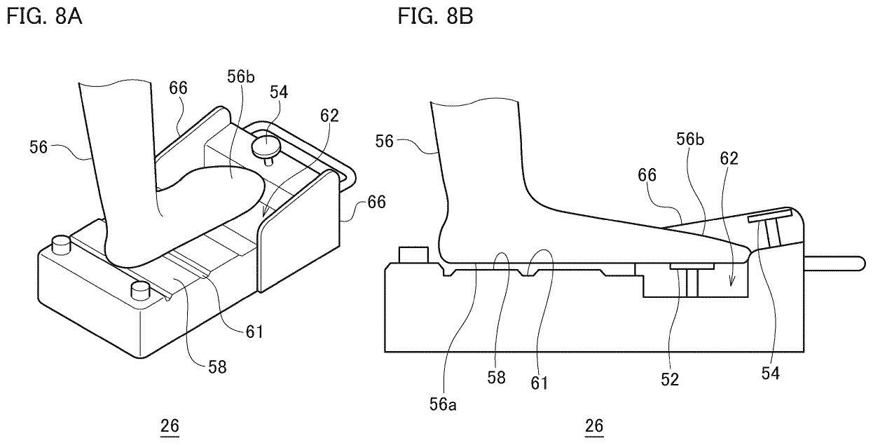

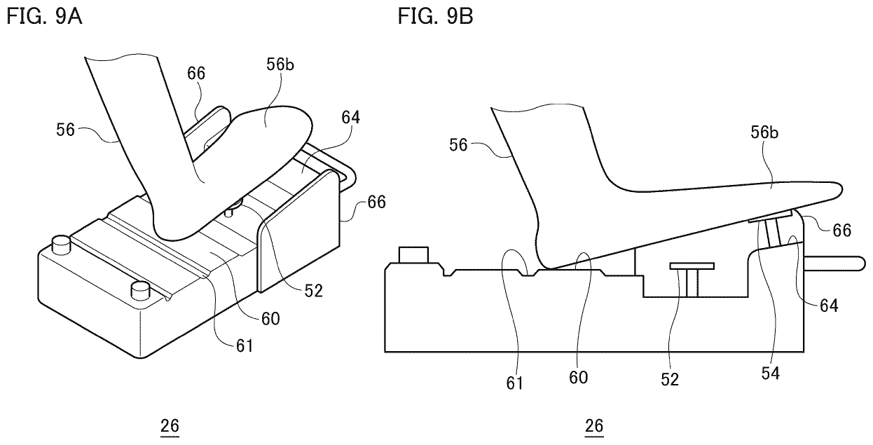

FIG. 8A is a perspective view of the foot controller in a state in which a foot is placed at the first position of the placement section according to some embodiments, and FIG. 8B is a side view of the foot controller in the state in which the foot is placed at the first position of the placement section according to some embodiments. FIG. 9A is a perspective view of the foot controller in a state in which a foot is placed at the second position of the placement section according to seem embodiments, and FIG. 9B is a side view of the foot controller in a state in which the foot is placed at the second position of the placement section according to some embodiments. Note that illustration of the kick switch (to be described later) on the right side face of the foot controller is omitted in FIGS. 8B and 9B.

The placement section 50 may include a first footrest 58 on which the heel 56a of a foot 56 placed at the first position P1 is placed, a second footrest 60 on which the heel 56a of a foot 56 placed at the second position P2 is placed, and a recess 62 at which the second joystick 54 is located. A groove 61 may be formed between the first footrest 58 and the second footrest 60 so as to make it easier to recognize the position at which a foot is to be placed.

As illustrated in FIGS. 8A and 8B, the foot 56 placed at the first position P1 may operate the first joystick 52 with the bottom of the toes 56b. In this state, the second joystick 54 is located at a position that does not interfere with the foot 56 placed at the first position P1. In other words, the second joy stick 54 may be located at a position adjacent to the foot 56 placed at the first position P1. As a result, it is unlikely that the second joystick 54 is erroneously touched while the first joystick 52 is being operated with the foot 56.

According to the foot controller 26 according to some embodiments, the second joystick 54 is located at a position higher than the first joystick 52. The higher position may be an elevated position. In addition, a region of the placement section 50 in which the second joystick 54 is located may include a tilted surface 64 with respect to an installation surface on which the foot controller 26 is installed, and the angle between the installation surface and the tilted surface 64 is α° (see FIG. 7B). Note that α° is an angle between 100 and 15°. In addition, a main surface of the placement section 50 and the installation surface are substantially parallel to each other.

As a result, as illustrated in FIGS. 9A and 9B, the foot 56 placed at the second position P2 has a toe-up posture and thus can operate the second joystick 54 with the bottom of the toes 56b. In this state, the first joystick 52 may be located at the bottom of the recess 62 so that the first joystick 52 does not interfere with the foot 56 placed at the second position P2. In other words, the first joystick 52 may be located at a position adjacent to the foot 56 placed at the second position P2. As a result, it is unlikely that the first joystick 52 is erroneously touched while the second joystick 54 is being operated with the foot 56.

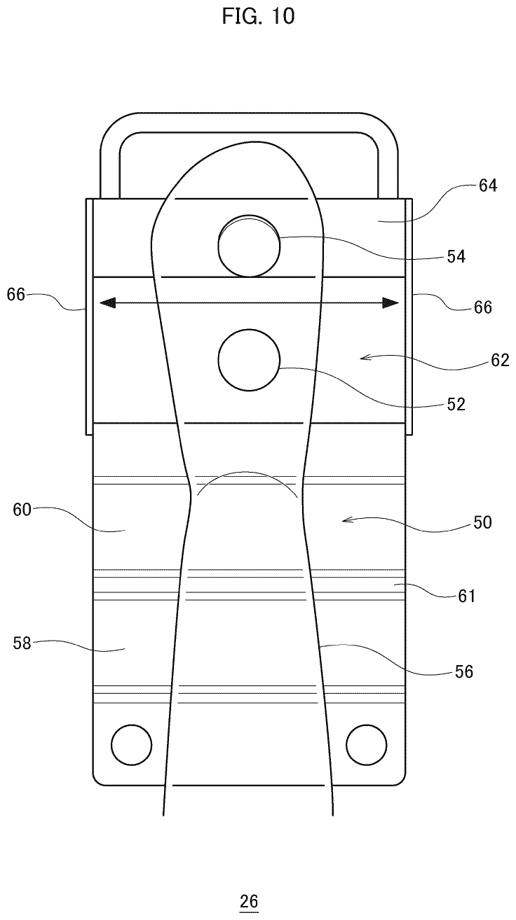

FIG. 10 is a top view of the foot controller in a state in which a foot is placed on the placement section according to some embodiments. The foot controller 26 may further include a kick switch 66, which is a third operating part, that is provided on a side of the placement section 50. As illustrated in FIG. 10, the kick switch 66 may be capable of being operated by a horizontal movement (in the direction of an arrow) of the foot 56 placed at the first position P1 and is also capable of being operated by a horizontal movement of the foot 56 placed at the second position P2. Thus, the kick switch 66 can be operated by a horizontal movement of the foot 56 both in the case where the foot 56 is placed at the first position P1 and in the case where the foot 56 is placed at the second position P2.

In some embodiments, the kick switch 66 may be provided in a form of a pair of kick switches 66 at respective sides of the placement section 50, and the first joystick 52 and the second joystick 54 may be arranged in a region between the pair of kick switches 66. The pair of kick switches 66 may be on the sides of the placement section corresponding to the left and right sides of the users foot Thus, the kick switch 66 can be operated by a horizontal movement of the foot 56 in either direction, which enables quick operation without a sense of discomfort as compared with a case where a kick switch is provided only on one side of the placement section 50 even if there is a difference in easiness of horizontal movement of a foot or a difference in easiness between a rightward movement and a leftward movement of a foot depending on the operator's dominant foot. This allows the foot controller 26 to be operated by both of left and right feet.

### [Operation of Endoscope Holder by Foot Controller]

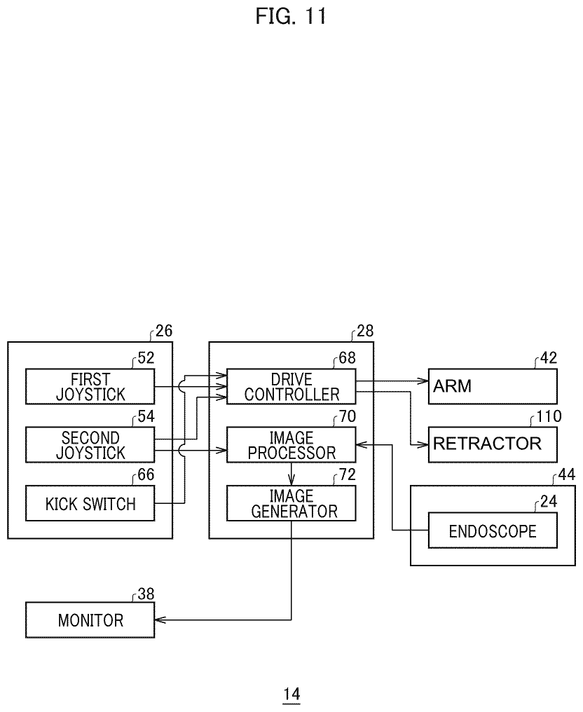

FIG. 11 is a block diagram of the surgery assistance device 14 according to some embodiments. As described above, the surgery assistance device 14 according to some embodiments may include the endoscope 24, the holder 44 for holding the endoscope 24, the arm 42 for adjusting the position of the holder 44, the retractor 110, the monitor 38 for displaying a visual field image taken by the endoscope 24, the foot controller 26, and the controller 28 that changes the visual field image 46 in accordance with operations performed on the foot controller 26.

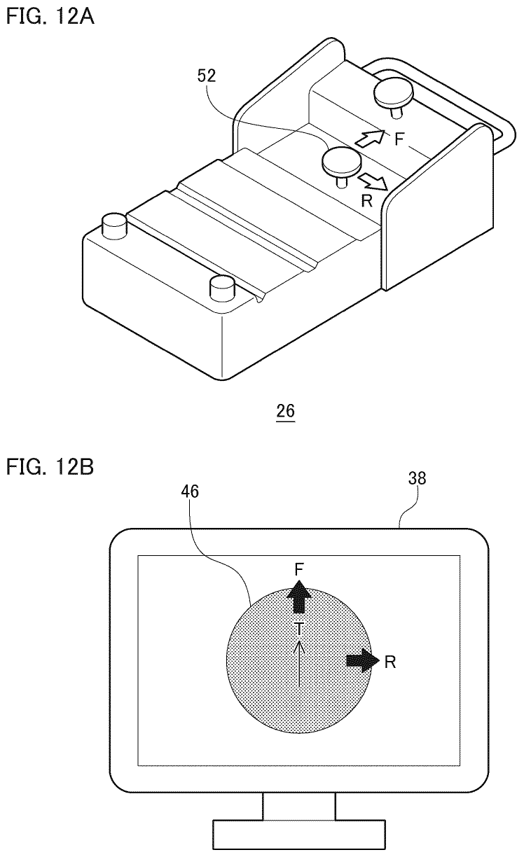

FIG. 12A is a schematic diagram illustrating an operation of a first joystick according to some embodiments, and FIG. 12B is a schematic diagram of a visual field image corresponding to the operation of the first joystick illustrated in FIG. 12A. In order to move the visual field on the visual field image 46 back and forth and left and right, a possible way is to change the orientation of the tip of the insertion portion 24a of the endoscope 24 back and forth and left and right.

Thus, a drive controller 68 included in the controller 28 may control the movement of the endoscope 24 through the arm 42 so that the visual field of the endoscope 24 in accordance with back and forth and left and right operations performed on the first joystick 52. For example, as illustrated in FIG. 2, the visual field of the visual field image 46 can be moved by tilting the endoscope 24 back and forth and left and right around a position at which the endoscope 24 is inserted into the eyeball 30. Thus, because the visual field image 46 may be changed in accordance with the operation of the first joystick 52 on the foot controller 26, interruption of manual work of the doctor 18, who is the operator, to change the visual field image 46 can be avoided.

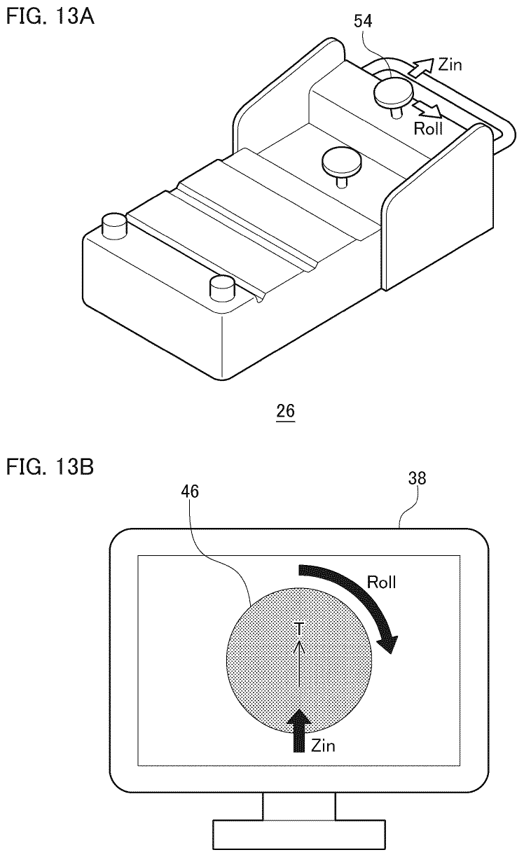

FIG. 13A is a schematic diagram illustrating an operation of a second joystick according to some embodiments, and FIG. 13B is a schematic diagram of a visual field image corresponding to the operation of the second joystick illustrated in FIG. 13A. For zooming in and zooming out of the visual field on the visual field image 46, a possible way is to advance and retract the endoscope 24 with respect to the eyeball 30.

Thus, the drive controller 68 of the controller 28 controls the movement of the endoscope 24 through the arm 42 so that the visual field of the endoscope 24 zooms in and out in accordance with the back and forth operation of the second joystick 54. For example, as illustrated in FIG. 2, the position of the endoscope 24 may be advanced and retracted (Zin, Zout) to cause the visual field image 46 to zoom in and out.

In addition, an image processor 70 included in the controller 28 may perform image processing so that the visual field image 46 turns in accordance with left and right operations of the second joystick 54. The visual field image resulting from image processing may be displayed on the monitor 38 via an image generator 72.

Thus, operation of the second joystick 54 is only needed to cause the visual field to zoom in and zoom out without regard to the movement of the endoscope 24, and the visual field image 46 can be turned by image processing without moving the endoscope 24. In other words, because the visual field image 46 can be changed in accordance with the operation of the second joystick 54 on the foot controller 26, interruption of manual work of the doctor 18, who is the operator, to change the visual field image 46 can be avoided.

### [Operation in Emergency]

When the kick switch 66 of the foot controller 26 is operated in an emergency, the surgery assistance device 14 according to some embodiments may be capable of retracting the endoscope 24 from the eyeball 30 by the retractor 110 described above. In other words, in an emergency, the doctor 18 only needs to move his/her foot 56 horizontally to retract the endoscope 24 from the eyeball 30.

While various embodiments have been described above with reference to the drawings, the present disclosure is not limited thereto, and any combination or substitution of components as appropriate is included within the scope of the present disclosure. In some embodiments, modifications such as combinations, changes in the order of processes, and various changes in design may be made on the basis of knowledge of a person skilled in the art, and such modified embodiments are within the scope of the present disclosure and the appended claims.

Embodiments consistent with the present disclosure can be used for a foot controller used to operate and control medical surgical tools during surgery.

What is claimed is:

1. A foot controller for operating a device, the foot controller comprising:
a placement section on which a foot is to be placed;
a first operating part configured to be moved back and forth and left and right by the foot placed at a first position of the placement section; and
a second operating part configured to be moved back and forth and left and right by the foot placed at a second position of the placement section,
wherein the first operating part and the second operating part are arranged along a front-back direction on the placement section, and
wherein the first operating part and the second operating part are further configured to select a predetermined operation of the device based on a movement of the foot.

2. The foot controller according to claim 1, wherein the first operating part is located at a recess in the placement section, and
wherein the second operating part is located at a position adjacent to the foot placed at the first position.

3. The foot controller according to claim 2, wherein the second operating part is located on the placement section at an elevated position from the first operating part, and
wherein the first operating part is located at a position adjacent to the foot placed at the second position.

4. The foot controller according to claim 1, further comprising:
a third operating part on a side of the placement section,
wherein the third operating part is configured to be operated by a horizontal movement of the foot placed at the first position or by the horizontal movement of the foot placed at the second position.

5. The foot controller according to claim 4, wherein the third operating part is a pair of switches on respective sides of the placement section, and
wherein the first operating part and the second operating part are located in a region between the pair of switches.

6. The foot controller according to claim 1, wherein the first operating part and the second operating part are joysticks.

7. A surgery assistance device comprising:
an endoscope comprising a camera;
a holder configured to hold the endoscope;
an arm configured to adjust a position of the holder;
a display configured to display a visual field image taken by the camera;
the foot controller according to claim 1; and
a controller configured to change the visual field image based on an operation on the foot controller.

8. The surgery assistance device according to claim 7, wherein the controller comprises a drive controller configured to move the endoscope in a back and forth direction and left and right direction, through the arm, corresponding to a movement of the first operating part.

9. The surgery assistance device according to claim 7, wherein the controller comprises:
a drive controller configured to advance and retract the endoscope, through the arm, corresponding to a back and forth movement of the second operating part by the foot; and
an image processor configured to perform image processing to rotate the visual field image corresponding to a left and right movement of the second operating part by the foot.

10. A surgery assistance device comprising:
an endoscope;
a holder configured to hold the endoscope;
an arm configured to adjust a position of the holder;
a retractor configured to retract the endoscope from an eyeball; and
the foot controller according to claim 4,
wherein the retractor is configured to retract the endoscope from the eyeball corresponding to the third operating part on the foot controller being operated.

11. The foot controller according to claim 1, wherein the placement section has a length less than 400 mm in the front-back direction.

12. The foot controller according to claim 1, wherein the placement section has a width less than 250 mm.

13. The foot controller according to claim 1, wherein the placement section has a height greater than 100 mm and less than 150 mm at a position having a highest elevation.

14. The foot controller according to claim 3, wherein the elevated position of the placement section is tilted at an angle greater than 10° and less than 15°.

15. The surgery assistance device according to claim 9, wherein the visual field image of the endoscope is configured to be zoomed in and out of in accordance to the back and forth movement of the second operating part by the foot.

16. A foot controller for operating a device, the foot controller comprising:
a placement section on which a foot is to be placed;
a first joystick configured to be moved back and forth and left and right by the foot placed at a first position of the placement section; and
a second joystick configured to be moved back and forth and left and right by the foot placed at a second position of the placement section,
wherein the first joystick and the second joystick are arranged along a front-back direction on the placement section, and
wherein the first joystick and the second joystick are further configured to select a predetermined operation of the device based on a movement of the foot.

17. The foot controller according to claim 16, wherein the first joystick is located at a recess in the placement section, the first joystick being adjacent to the foot placed at the second position, and
wherein the second joystick is located on the placement section at an elevated position from the first joystick, the second joystick being adjacent to the foot placed at the first position.

18. The foot controller according to claim 16, further comprising:
a pair of switches on respective sides of the placement section,
wherein the pair of switches are configured to be operated by a horizontal movement of the foot placed at the first position or by the horizontal movement of the foot placed at the second position, and
wherein the first joystick and the second joystick are located in a region between the pair of switches.

19. A surgery assistance device comprising:
an endoscope comprising a camera;
a holder configured to hold the endoscope;
an arm configured to adjust a position of the holder;
a display configured to display a visual field image taken by the camera;
the foot controller according to claim 16; and
a controller configured to change the visual field image based on an operation on the foot controller,
wherein the controller comprises a drive controller configured to move the endoscope in a back and forth direction and left and right direction, through the arm, corresponding to a movement of the first joystick,
wherein the controller comprises:
a drive controller configured to advance and retract the endoscope, through the arm, corresponding to a back and forth movement of the second joystick by the foot; and
an image processor configured to perform image processing to rotate the visual field image corresponding to a left and right movement of the second joystick by the foot, and
wherein the visual field image of the endoscope is configured to be zoomed in and out of in accordance to the back and forth movement of the second joystick by the foot.

20. A surgery assistance device comprising:
an endoscope;
a holder configured to hold the endoscope;
an arm configured to adjust a position of the holder;
a retractor configured to retract the endoscope from an eyeball; and
the foot controller according to claim 18,
wherein the retractor is configured to retract the endoscope from the eyeball corresponding to the pair of switches on the foot controller being operated.
